(19) United States
(12) Patent Application Publication
(10) Pub. No.: US 2019/0103171 A1
(43) Pub. Date: Apr. 4, 2019

(54) CONFIGURABLE WORKFLOW FOR PATHOLOGY LABS

(71) Applicant: Cerebrum Corporation, Chandler, AZ (US)

(72) Inventors: Gregg Lahti, Gilbert, AZ (US); Matthew Hoppes, Phoenix, AZ (US); Michael Howell, Chandler, AZ (US); Brandon Sleater, Gilbert, AZ (US)

(21) Appl. No.: 16/142,390

(22) Filed: Sep. 26, 2018

Related U.S. Application Data
(60) Provisional application No. 62/565,329, filed on Sep. 29, 2017.

(51) Int. Cl. G16H 10/40

(52) U.S. Cl. CPC G16H 10/40

(57) ABSTRACT

Computing systems and computer-implemented methods for managing pathology lab workflow include storing a plurality of system objects, each system object representing an item to be tracked in the pathology lab workflow, the plurality of system objects including objects selected from the group consisting of accession, patient, and tissue samples. A plurality of system object maps are also stored, each system object map designating transitions between operations being tracked within the pathology lab workflow. The method also includes performing a multi-relational analysis of two or more system objects applied to one or more system object maps to identify a next state in the pathology lab for an item being tracked and outputting to a user the next state in the pathology lab for the item being tracked.

### CROSS-REFERENCE TO RELATED APPLICATION

This application claims the benefit under 35 U.S.C. § 119(e) of U.S. Provisional Patent Application Serial No. 62/565,329, filed Sep. 29, 2017, the disclosure of which is hereby incorporated herein in its entirety by this reference.

This application is also related to U.S. Patent Application Serial No. TBD, filed concurrently with this application and entitled “Macro-based Diagnoses for Anatomic Pathology,” which claims the benefit under 35 U.S.C. § 119(e) of U.S. Provisional Patent Application Serial No. 62/565,320, filed Sep. 29, 2017, the disclosures of which are hereby incorporated herein in their entirety by this reference.

### TECHNICAL FIELD

Embodiments of the present disclosure relate generally to managing laboratory information and more specifically to the application of directing workflow by determining a configurable workflow process in an anatomic pathology laboratory.

### BACKGROUND

In an anatomic pathology lab, human or animal tissue is processed through various methods to achieve a thin slice of stained tissue on a slide. In general, the operational work done on a specimen goes from one step or station to another in a sequence. These steps are related to the work being done on the specimen to create a final product of the stained tissue on the slide. Significant time and productivity is lost when this movement through the various laboratory operations is managed inefficiently. However, because the permutations of possible workflow and inventory of items in a lab are so large, it is very difficult to create efficient workflows by current conventional processes.

### BRIEF DESCRIPTION OF THE DRAWINGS

FIG. 1 illustrates a computing system for practicing embodiments of the present disclosure.

FIG. 2 shows a screenshot of the software application setup for an anatomic pathology lab workflow.

FIG. 3 shows a screenshot of the software application showing a list of various System Objects that may be included in a pathology lab workflow.

FIG. 4 shows a screenshot associated with a specific patient that may be included in a pathology lab workflow.

FIG. 5 shows an example screenshot of editing an ENTITY System Object.

FIG. 6 shows an example list of System Object Maps that may be utilized within the software application.

FIG. 7 shows a partial list of a System Object Map.

FIG. 8 shows a partial definition of a STDPROC_BASE System Object Map.

FIG. 9 is a flow diagram illustrating a workflow evaluation process.

FIG. 10 illustrates portions of a single-layer map structure.

FIG. 11 illustrates portions of a multi-layer map structure.

### DETAILED DESCRIPTION

In the following description, reference is made to the accompanying drawings in which is shown, by way of illustration, specific embodiments in which the disclosure may be practiced. The embodiments are intended to describe aspects of the disclosure in sufficient detail to enable those skilled in the art to practice the invention. Other embodiments may be utilized and changes may be made to the disclosed embodiments without departing from the scope of the disclosure. The following detailed description is not to be taken in a limiting sense, and the scope of the present invention is defined only by the appended claims.

Furthermore, specific implementations shown and described are only examples and should not be construed as the only way to implement the present disclosure unless specified otherwise herein. It will be readily apparent to one of ordinary skill in the art that the various embodiments of the present disclosure may be practiced by numerous other partitioning solutions.

In the following description, elements, circuits, and functions may be shown in block diagram form in order not to obscure the present disclosure in unnecessary detail. Conversely, specific implementations shown and described are exemplary only and should not be construed as the only way to implement the present disclosure unless specified otherwise herein. Additionally, block definitions and partitioning of logic between various blocks is exemplary of a specific implementation. It will be readily apparent to one of ordinary skill in the art that the present disclosure may be practiced by numerous other partitioning solutions. For the most part, details concerning timing considerations and the like have been omitted where such details are not necessary to obtain a complete understanding of the present disclosure and are within the abilities of persons of ordinary skill in the relevant art.

Those of ordinary skill in the art would understand that information and signals may be represented using any of a variety of different technologies and techniques. For example, data, instructions, commands, information, signals, bits, symbols, and chips that may be referenced throughout this description may be represented by voltages, currents, electromagnetic waves, magnetic fields or particles, optical fields or particles, or any combination thereof. Some drawings may illustrate signals as a single signal for clarity of presentation and description. It will be understood by a person of ordinary skill in the art that the signal may represent a bus of signals, wherein the bus may have a variety of bit widths and the present disclosure may be implemented on any number of data signals including a single data signal.

The various illustrative logical blocks, modules, and circuits described in connection with the embodiments disclosed herein may be implemented or performed with a general purpose processor, a special purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A general-purpose processor may be considered a special-purpose processor while the general-purpose processor is configured to execute instructions (e.g., software code) related to embodiments of the present disclosure. A processor may also be implemented as a combination of computing devices, such as a combination of a DSP and a microprocessor, a plurality of microprocessors, one or more microprocessors in conjunction with a DSP core, or any other such configuration.

Also, it is noted that the embodiments may be described in terms of a process that is depicted as a flowchart, a flow diagram, a structure diagram, or a block diagram. Although a flowchart may describe operational acts as a sequential process, many of these acts can be performed in another sequence, in parallel, or substantially concurrently. In addition, the order of the acts may be re-arranged. A process may correspond to a method, a thread, a function, a procedure, a subroutine, a subprogram, etc. Furthermore, the methods disclosed herein may be implemented in hardware, software, or both. If implemented in software, the functions may be stored or transmitted as one or more instructions or code on computer-readable media. Computer-readable media includes both computer storage media and communication media including any medium that facilitates transfer of a computer program from one place to another.

In some embodiments, some or all of the features described herein are implemented within a computer processor or processing device that executes software procedures. The transformation of data that occurs according to the specific procedures of embodiments described herein render the processing device executing such embodiments as a special-purpose processing device capable of new functionality that is otherwise not available executing conventional software or logical procedures. In addition, efficient processing of such procedures requires implementation within computer processing systems. Furthermore, the interactions between an electronic storage device to store data associated with the techniques described herein and the computer processing devices to execute the techniques described herein achieve much greater efficacy than would be possible through other non-computerized means.

For at least these reasons, various embodiments of the present disclosure, as described more fully herein, provide a technical solution to one or more problems that arise from technology that could not reasonably be performed by a person, and various embodiments disclosed herein are rooted in computer technology in order to overcome the problems and/or challenges described below. Further, at least some embodiments disclosed herein may improve computer-related technology by allowing computer performance of a function not previously performable by a computer.

It should be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed there or that the first element must precede the second element in some manner. In addition, unless stated otherwise, a set of elements may comprise one or more elements.

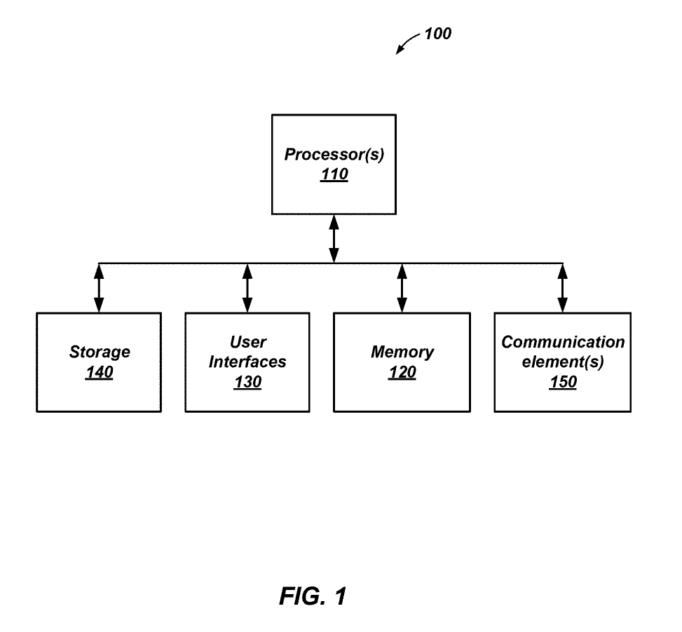

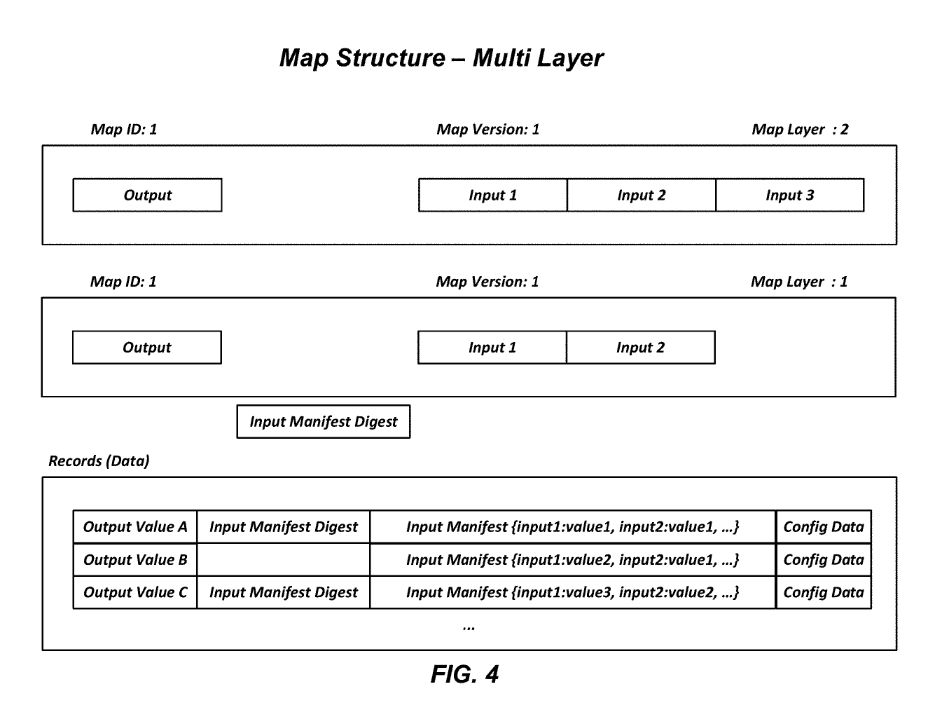

Elements described herein may include multiple instances of the same element. These elements may be generically indicated by a numerical designator (e.g. 110) and specifically indicated by the numerical indicator followed by an alphabetic designator (e.g., 110A) or a numeric indicator preceded by a “dash” (e.g., 110-1). For ease of following the description, for the most part element number indicators begin with the number of the drawing on which the elements are introduced or most fully discussed. Thus, for example, element identifiers on a FIG. 1 will be mostly in the numerical format 1xx and elements on a FIG. 4 will be mostly in the numerical format 4xx.

Headings may be included herein to aid in locating certain sections of detailed description. These headings should not be considered to limit the scope of the concepts described under any specific heading. Furthermore, concepts described in any specific heading are generally applicable in other sections throughout the entire specification.

Reference throughout this specification to “one embodiment,” “an embodiment,” or similar language means that a particular feature, structure, or characteristic described in connection with the indicated embodiment is included in at least one embodiment of the present disclosure. Thus, the phrases “in one embodiment,” “in an embodiment,” and similar language throughout this specification may, but do not necessarily, all refer to the same embodiment.

Before describing specific embodiments, and in order to facilitate description in the present disclosure, various terms are described herein. Where ambiguity may exist between the plain meaning, dictionary meaning, and the term as described herein, a person of ordinary skill in the art will recognize the term as described herein will best conform to a more comprehensive understanding of embodiments of the present disclosure.

As used herein, unless referred to specifically with a different meaning, an “accession” is a test or group of tests ordered for a particular specimen received by a lab or other health care service.

As used herein the term “module” means a software process, a collection of software processes, a collection of hardware elements, or a combination thereof configured to implement one or more elements of the present disclosure

As used herein, the term “substantially” in reference to a given parameter, property, or condition means and includes to a degree that one of ordinary skill in the art would understand that the given parameter, property, or condition is met with a small degree of variance, such as, for example, within acceptable manufacturing tolerances. By way of example, depending on the particular parameter, property, or condition that is substantially met, the parameter, property, or condition may be at least 90% met, at least 95% met, or even at least 99% met.

Some drawings presented herein include depictions of a Graphical User Interface (GUI), which may include color elements. Embodiments of the present disclosure address issues such as lab accuracy and lab efficiency. As such, formatting and colors associated with certain elements may be useful for increasing efficiency and accuracy by assisting a user with performing tasks related to presentation and modification of information related to embodiments of the present disclosure.

FIG. 1 illustrates a computing system 100 for practicing embodiments of the present disclosure. The computing system 100 may be a user-type computer, a file server, a compute server, or other similar computer. Computer, computing system, and server may be used interchangeably herein to indicate a system for practicing embodiments of the present disclosure. The computing system 100 is configured for executing software programs containing computing instructions and includes one or more processors 110, memory 120, one or more communication elements 150, user interface elements 130, and storage 140.

As non-limiting examples, the computing system 100 may be a user-type computer, a file server, a compute server, a notebook computer, a tablet, a handheld device, a mobile device, or other similar computer system for executing software.

The one or more processors 110 may be configured for executing a wide variety of operating systems and applications including the computing instructions for carrying out embodiments of the present disclosure.

The memory 120 may be used to hold computing instructions, data, and other information for performing a wide variety of tasks including performing embodiments of the present disclosure. By way of example, and not limitation, the memory 120 may include Synchronous Random Access Memory (SRAM), Dynamic RAM (DRAM), Read-Only Memory (ROM), Flash memory, and the like.

Information related to the computing system 100 may be presented to, and received from, a user with one or more user interface elements. As non-limiting examples, the user interface elements may include elements such as displays, keyboards, mice, joysticks, haptic devices, microphones, speakers, cameras, and touchscreens. A display on the computing system may be configured to present a GUI with information about the embodiments of the present disclosure, as is explained below.

The communication elements 150 may be configured for communicating with other devices or communication networks. As non-limiting examples, the communication elements 150 may include elements for communicating on wired and wireless communication media, such as for example, serial ports, parallel ports, Ethernet connections, universal serial bus (USB) connections IEEE 1394 (“firewire”) connections, Bluetooth wireless connections, 802.1 a/b/g/n type wireless connections, and other suitable communication interfaces and protocols.

The storage 140 may be used for storing relatively large amounts of non-volatile information for use in the computing system 100 and may be configured as one or more storage devices. By way of example, and not limitation, these storage devices may include computer-readable media (CRM). This CRM may include, but is not limited to, magnetic and optical storage devices such as disk drives, magnetic tapes, CDs (compact disks), DVDs (digital versatile discs or digital video discs), and other equivalent storage devices.

Software processes illustrated herein are intended to illustrate representative processes that may be performed by the systems illustrated herein. Unless specified otherwise, the order in which the process acts are described is not intended to be construed as a limitation, and acts described as occurring sequentially may occur in a different sequence, or in one or more parallel process streams. It will be appreciated by those of ordinary skill in the art that many steps and processes may occur in addition to those outlined in flow charts. Furthermore, the processes may be implemented in any suitable hardware, software, firmware, or combinations thereof.

When executed as firmware or software, the instructions for performing the processes may be stored on a computer-readable medium. A computer-readable medium includes, but is not limited to, magnetic and optical storage devices such as disk drives, magnetic tape, CDs (compact disks), DVDs (digital versatile discs or digital video discs), and semiconductor devices such as RAM, DRAM, ROM, EPROM, and Flash memory.

By way of non-limiting example, computing instructions for performing the processes may be stored on the storage 140, transferred to the memory 120 for execution, and executed by the processors 110. The processors 110, when executing computing instructions configured for performing the processes, constitutes structure for performing the processes and can be considered a special-purpose computer when so configured. In addition, some or all portions of the processes may be performed by hardware specifically configured for carrying out the processes.

Using a computing system 100 with a graphical operating system, embodiments of the present disclosure include software application tools providing functional panels or windows within the application to segment the functionality. Many panels can be used to describe multiple functions but in this description two of the panels will be presented to the user which control and display the items being utilized within the application tool. The application tool is used to create digital content for various products of which one element could be to generate a GUI to run on a computing system 100 utilizing an LCD touch/display screen. In this application tool, the user provides graphical data in the form of images, pictures, and text to create a digital content output. The digital content output could be represented on a computer screen or hardware screen or output to paper.

In an anatomic pathology laboratory operation, the operational work done on a specimen goes from one step or station to another in a sequence. These steps are related to the work being done on the specimen to create a final product of a stained tissue on a slide that can be presented to the pathologist for reading. Embodiments of the present disclosure include a software application to track each specimen on where it is in the lab workflow process and what step it should go to next. There are several variables that weigh into such decisions, for example: what type of specimen it is and what types of tests need to be performed for final output. To do this, the software application utilizes at least two processing identification items (IDs) within the system: a System Object (SO) and a System Object Map (SOM). These two IDs provide a multi-relational system to qualify the item or thing (System Object) that needs to be operated on and the location within the lab (System Object Map) where the item is presently located, where it needs to go next, or a combination of the two. The System Object Map provides a method that defines connections between two or more entities identified with a use context. These two items (System Objects and System Object Maps) are used to create many-to-many relationships which are then used within the software application for specimen management. The software application utilizes several items (e.g., a database, filesystem, application memory) to provide persistent storage for the System Objects and System Object Maps.

For items being stored in a database, the software application utilizes a relational database system with a set of tables to store the structures. There are different elements that may be defined in the database within the RDBMS (Relational Data Base Management System) schema. Some of these items can be listed as follows:
System ENUM: These are system-wide global key-value-pair values which are independent of all other data within the database schema.
System Object: Examples can be Orders, Order Items, Tests, Materials, etc.
System Object Attributes: These are sub-elements or properties of a System Object which do not directly relate to any other System Objects.
System Object Reference: These are sub-elements or properties of a System Object, which are relatable or linked to another System Object. For example, if implemented using a RDBMS these are Foreign Key columns in a table, that reference another System Object.

The software application uses data elements to provide a callback type and value as a return object to the calling function. The input data element allows what is stored in the map as a return value of the callback rather than the input values themselves. The map input for this data element becomes the input signature of the callback function itself, allowing for complex input value processing and evaluation functions such as range lookups, etc. Elements of this type are referenced in the map but are defined as callbacks in the application layer. The configuration value in the map is the return value of the callback (also classified as a Data Element). An example of this could be a Data Element return value of a function call operation which can be configured to a callback. The values configured in the map are the actual operations themselves, the callback (return object) of the function is “called” by the software application to obtain the value.

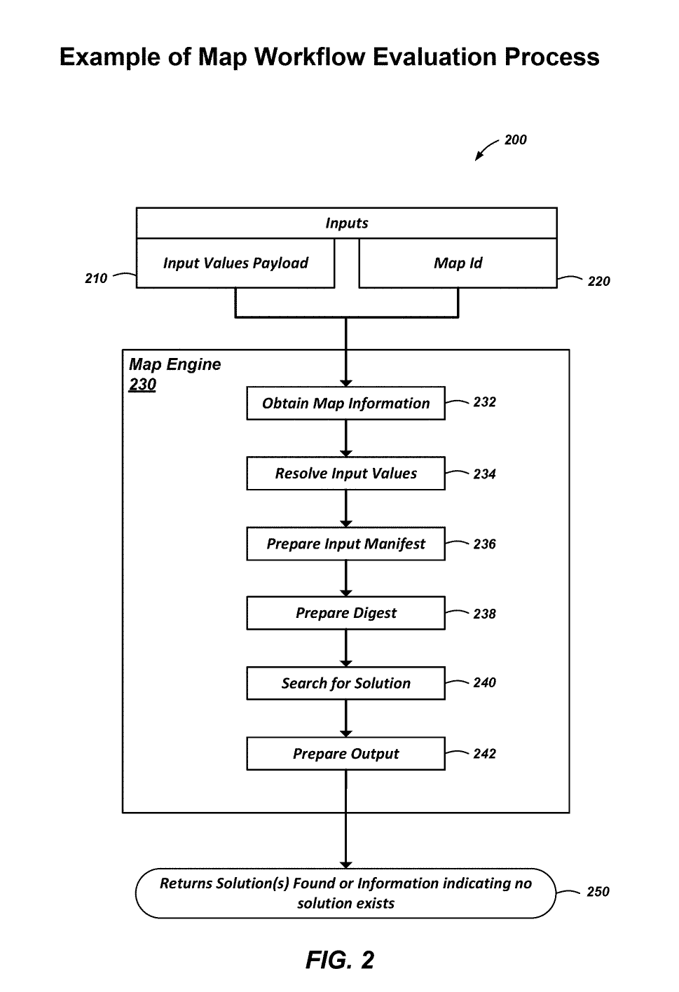

FIG. 2 is a flow diagram illustrating a workflow evaluation process. Process 200 shows the operation method of the software application when processing the System Object Maps. The software application uses the map stack (i.e., a stack of System Object Maps) to process the operation when the user identifies that the current workflow step is complete. Within the map engine 230, process(es) look at the appropriate input System Objects (usually workstation, tissue being operated, entity location of the lab, etc.) and then walk the stack of System Object Maps. The software application map engine compares the current IDs (System Objects) being used at the current workflow step and successively compares each of the System Object Map definitions in the collection to qualify if the assigned input IDs match the current IDs being used. Upon the first match, the output ID is used as the resultant determination of what step or operation to do next.

The software application utilizes a decision flow process to process these maps as shown in FIG. 2. The decision process uses an input values payload 210 presented along with a Map ID 220. At process blocks 232 and 234, the map engine 230 obtains map information, and resolves the input values used. At process blocks 236, the map engine 230 then formats the data into the appropriate key-value pairs and types presented. At process blocks 240, the map engine 230 then searches for a matching solution of the digest of input values presented. Upon a match, at process block 242, the map engine 230 prepares the output value from the map callback structure.

At process block 250, the software application returns the solution found and prepared from operation 242. If the map engine 230 does not find a solution, the map engine returns a non-solution value.

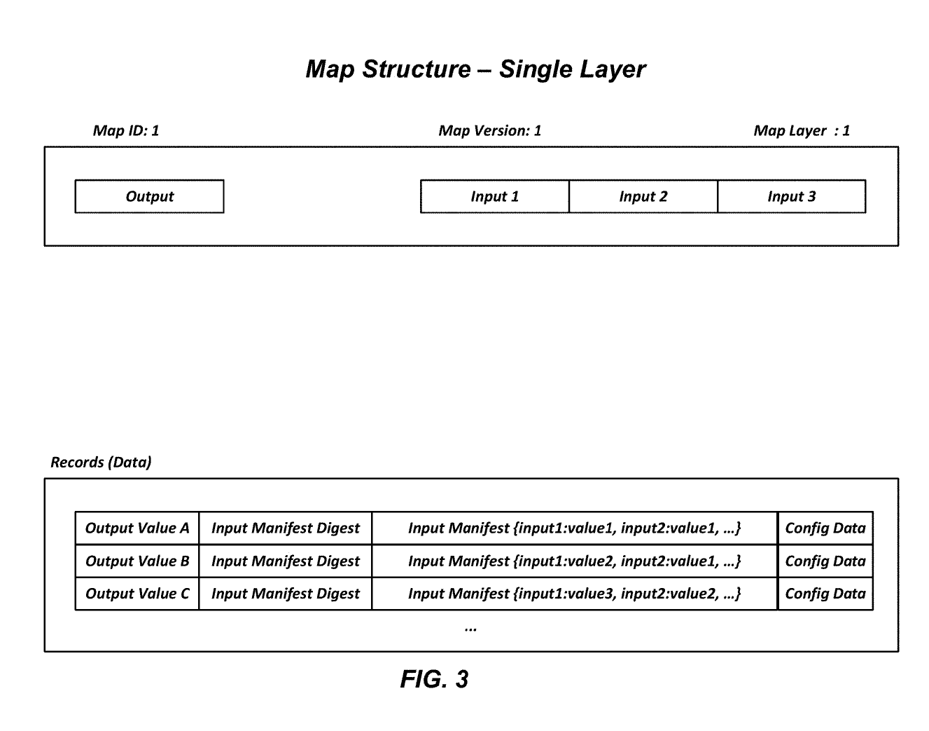

FIG. 3 illustrates portions of a single-layer map structure. This structure comprises a 1:1 relational map. For example, there are level 1 maps:

L1 1=>A

L1 2=>B

L1 3=>C

The relationship of the first layer one (L1) 1=>A is a 1:1 of the first item (1) to the second item (A), item (2) is related to B, etc. FIG. 3 shows a visual example for a single-layer map structure.

Conditional maps based on multiple input combinations of the same scope (different value combinations of the same input type, 1 layer) may be described as:

L1 1.1.2=>A

L1 1.2.1=>B

L1 2.3.2=>C

Complex maps based on multiple input combinations of varying scope (e.g., cascading multiple layers, until a solution is found), can be described as the following:

L3 2.3.4.5.1=>D

L2 1.2.3.1=>E

L2 1.2.3.3=>A

L1 1.1.2=>A

L1 1.2.1=>B

L1 2.3.2=>C

List Generation Mapping—single layer, can be described as:

L1 1.2=>A

L1 1.2=>B

L1 1.2=>C

Equivalency Mapping (Reverse List, single layer), can be described as

L1 1.1=>A

L1 1.2=>A

L1 1.3=>A

FIG. 4 illustrates portions of a multi-layer map structure within a database. Some points should be denoted in the single-layer and multi-layer map structure usage: A map including Resultant/Output can be another map, allowing for complex definitions/relationships.

Map evaluation information can be configured to define the evaluation process, for example:
Enable/Disable list type, means multiple match returns are ok.
Enable/Disable error on no solution, means a valid solution must always exist during evaluation or no solution exists.
Enable/Disable layer cascade continuation, means return on the LAST solution found from all layers evaluated as opposed to returning on the first solution found.

As another example, a workflow process step can be defined by describing the steps or operations based on input conditional values of using two maps. These two maps (e.g., A, B) can be defined as:
1. Map “A” is used to select a workflow process based on an input condition of consistent scope (only one layer)
2. Map “B” is used to define a workflow process based on an input condition set with variation (multiple layers)

Map “A” may be defined as:
Input :: Order Type|Input Service|Input :: Performing Location|Output Process|
   |Type 1|Service 1|Location 1|Process 1|
   |Type 1|Service 2|Location 1|Process 1|
   |Type 1|Service 1|Location 2|Process 2|

Map “B” may be defined as:
##### Layer 1
   |Input :: Current Step|Input :: Extra Condition|Output :: Next Step|
   |Step 2|Condition A|Step 4|
##### Layer 0
   |Input :: Current Step|Output :: Next Step|
   |---|---|
Step 0|Step 1|
Step 1|Step 2|
Step 2|Step 3|
Step 3|Step 4|

In the above examples, workflow steps can now be derived by defining input and output operations for each of the levels of map.

For many of the specimens, the sequence is predictable and similar. In some situations, the specimen type is special, a special test flow needs to be applied, or there are other input parameters that affect the lab operation that need to be addressed and route the specimen differently. Different labs will have one or more different special scenarios which cause extra work to be done compared to a normal workflow scenario of tissue and tests. To accommodate these extra variances within the workflow of a lab, the software application used to track the specimens in the lab needs to know about these variances.

The lab manager of the lab utilizes the software application to program the workflow map through a user interface to instruct the software application how to handle the exception cases that can occur. The software application uses these maps to determine all the work flow.

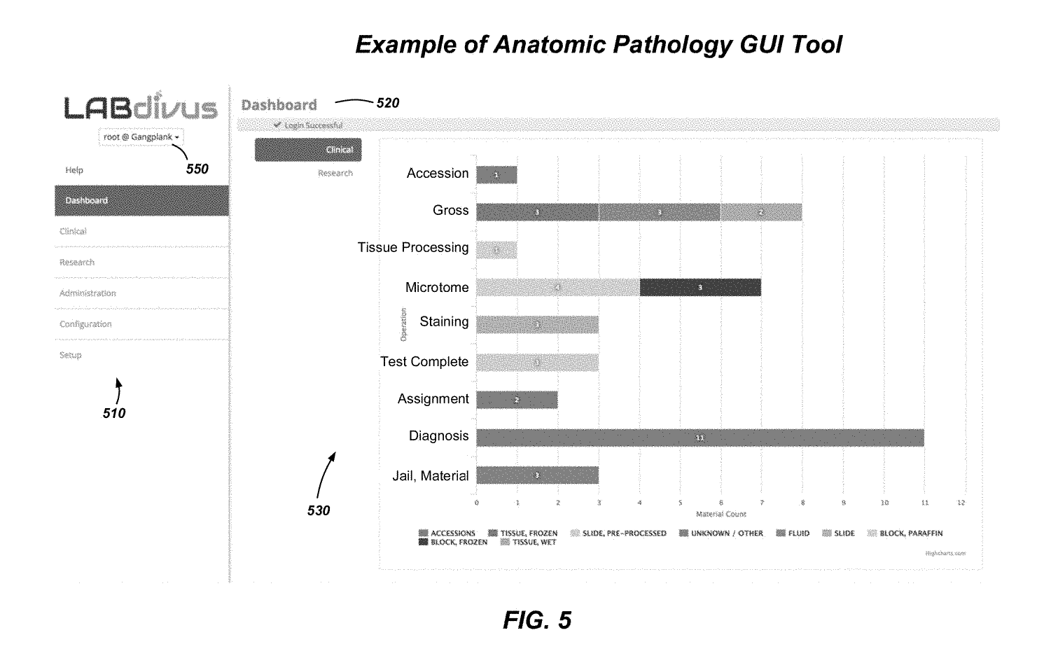

FIG. 5. shows a screenshot of the software application setup for an anatomic pathology lab workflow. A navigation indicator 520 is shown along the top side of the window with the text “Dashboard” displayed as the current navigation location. A user login indicator 550 shows the current user and may be configured as a selection element (e.g., a dropdown box) to change to a different user. A menu 510 shows the various operations that a user can perform from this window. An information window 530 illustrates various operations and elements, such as, for example, accession, tissue processing, microtome, and staining are show, as well as various processes within these operations.

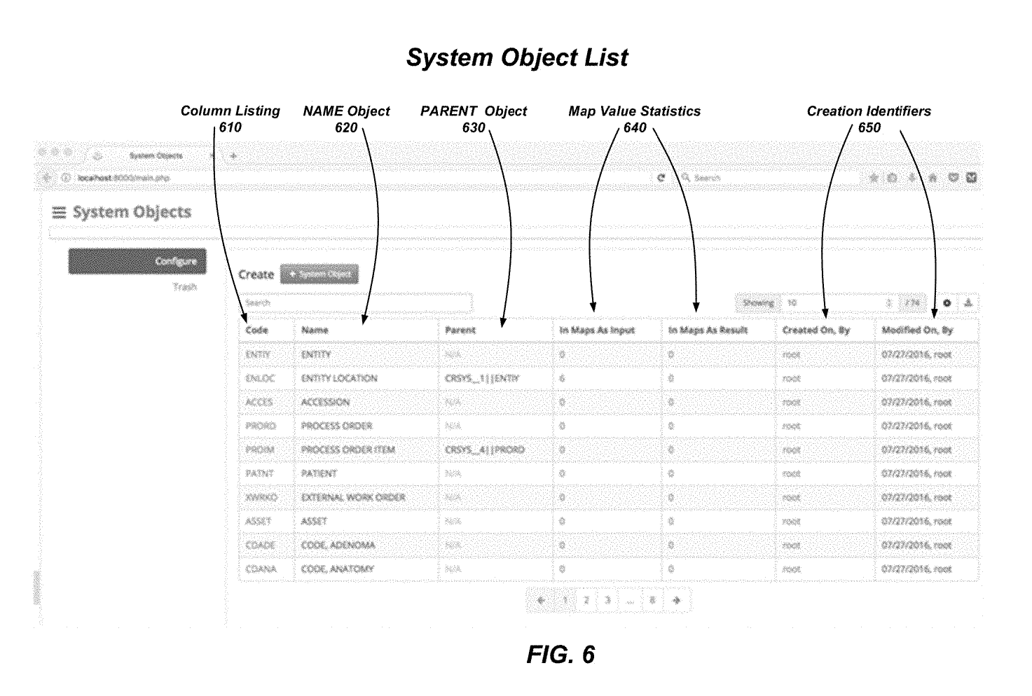

FIG. 6 shows a screenshot of the software application showing an example list of various System Objects that may be included in a pathology lab workflow. The table columns show the organization of attributes for each of the System Objects. The list shows the CODE column 610, which is the assigned code name of the System Object. That code is used within the software application to identify the System Object. The NAME column 620 is the named description of the CODE 610. The software application can identify if this is a PARENT System Object (i.e., it is the root of a material that children material could be created from) using the PARENT setting 630. The “In Maps As Input” and “In Maps As Result” columns 640 are statistical columns showing how many System Object Maps are currently referring to the particular System Object. For historical tracking purposes, the “Created On, By” and “Modified On, By” columns 650 designate who created the map, when the map was created, when the map was last modified, and who last modified the map.

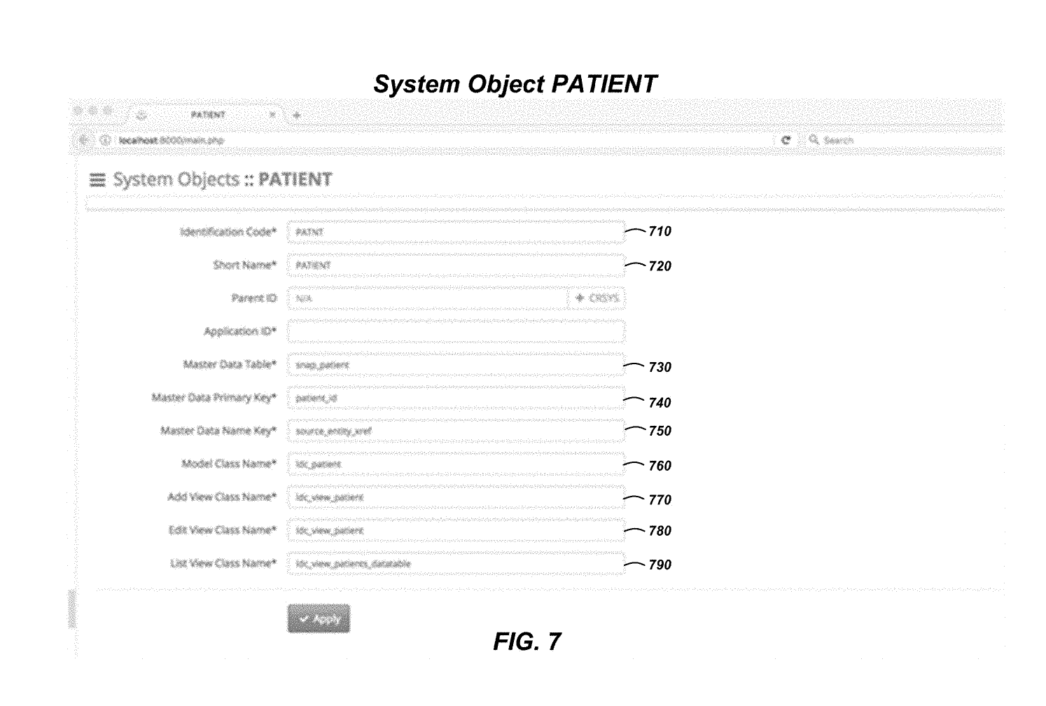

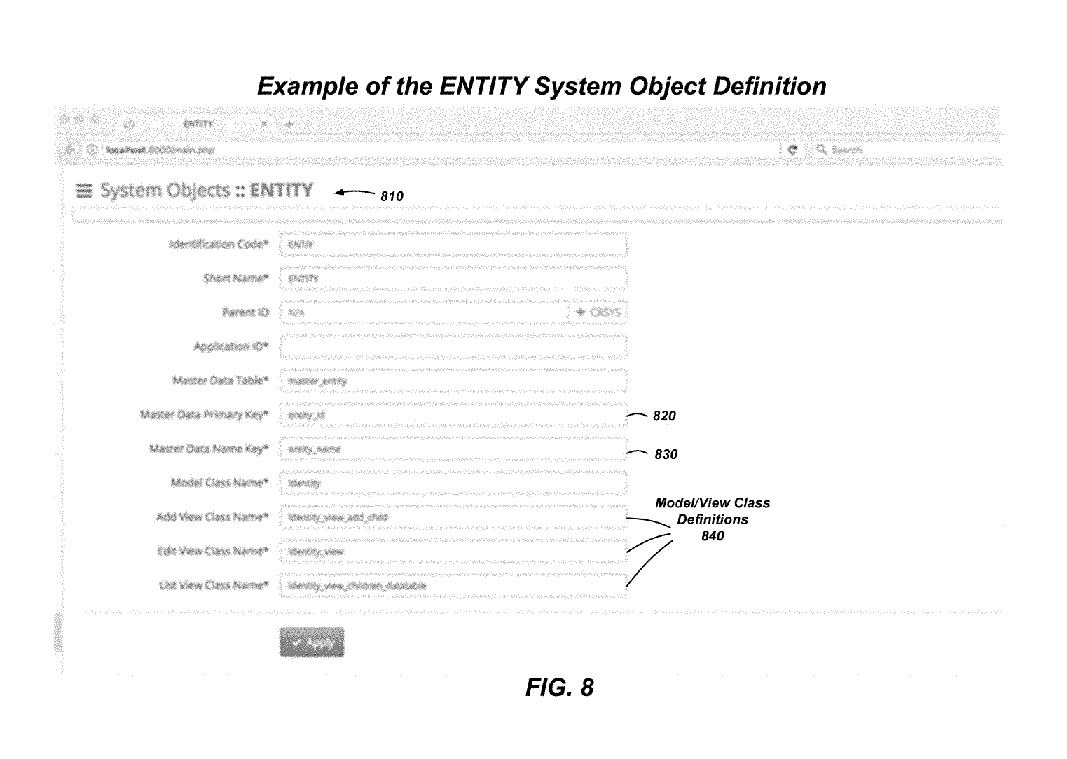

FIG. 7 shows a screenshot associated with a specific patient that may be included in a pathology lab workflow. In FIG. 7, a System Object of PATIENT is shown which may include the following attributes: an Identification Code Name 710, a short code word 720, a link to the master data type 730, a master data primary key 740, a master data name key 750, a model class name 760, an add view class name 770, an edit view class name 780, and a list view class name 790. These items help define the System Object within the software application, such as a patient, an asset, or a piece of equipment, a CPT code, an email address, a laboratory location, or a workstation (i.e. lab operation point of doing work), etc. The table and key fields establish a link into the database for the object and the class entries link the business functions that manipulate the object within the software application. The software application allows any N-number of System Objects to identify items utilized within the software application. The entries show a link into the software application database for the table and keys as well as the function classes called to operate on the patient object. FIG. 7 shows a specific example for a PATIENT object, which is used to describe a patient identified in the system. Other similar tables and entry items may be included for other System Objects and a person of ordinary skill in the art would be able to understand the construction and details of these other System Object based on the example presented in FIG. 7 for the patient System Object and an example for an ENTITY System Object shown in FIG. 8.

FIG. 8 shows an example screenshot of editing the ENTITY System Object. In FIG. 8 a full breakdown of what each object can be described as is shown. This is a raw input that shows the System Object and how it relates to the master data tables within the software application database and what software application code class name it is related to. The functional code classes that drives the GUI and software application business logic are referenced here. For the ENTITY object 810, which is a basic System Object provided in the software application, the master data primary key within the software application is called entity_id 820 and the master data name key is the entity_name 830. These are specific key names contained with the software application database. The Model and View classes 840 that control the GUI operations use the identity_view_add_child, identity_view, and identify_view_children_datatable classes 840 within the software application.

The software application then makes use of a System Object Map to relate the objects with each other.

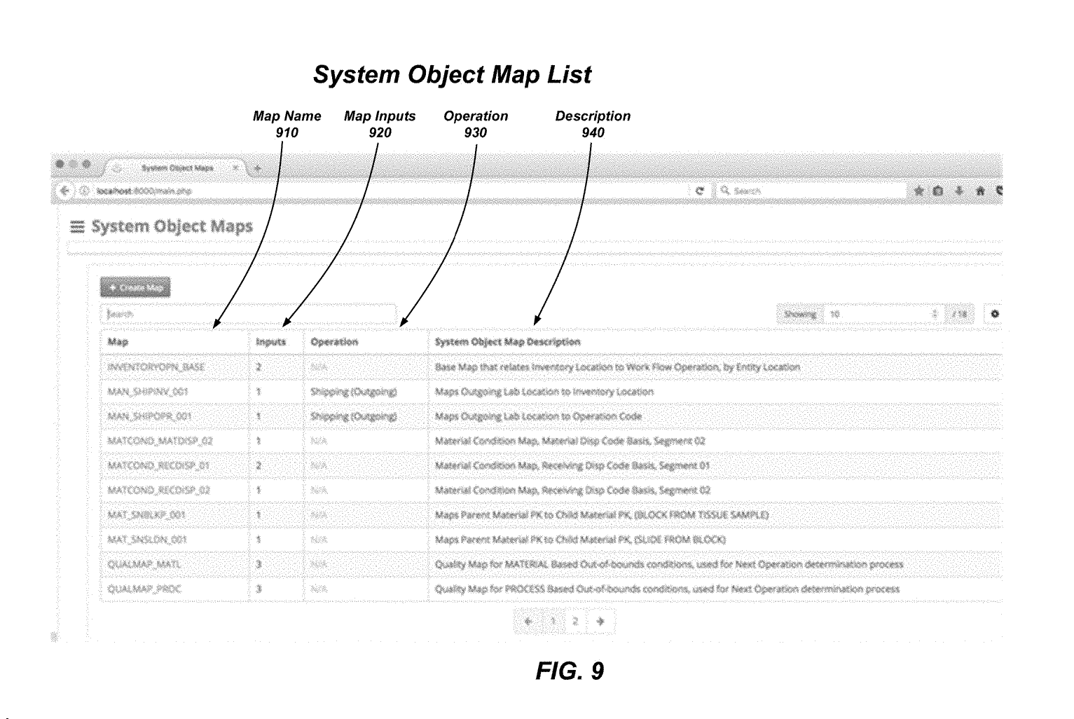

FIG. 9 shows an example list of System Object Maps that may be utilized within the software application. The screenshot includes entries for; the Map name 910, how many ID Inputs 920 are used for the map type, the Operation name 930 if applicable, and the System Object Map Description 940 that describes what the map is used for. These maps are referenced as part of the business logic of the software application to direct the processing of the specimen through the workflow in the lab. Each map has a variable number of input IDs and one output result ID. Each ID is a representative System Object allowing a mixture of any type of System Object as an input type. The output type is another System Object. Various System Object Maps can be created with variable input IDs but will generally have one output ID. In the software application, the maps used are usually 2 or 3 inputs, but could be expanded to say 7 or 10 inputs depending upon the complexity needed for the input relationships. Each of the maps in the collection are used to process work being done or establish a relationship of the System Objects. One example could be how a specimen moves from the accession stage to the gross stage in the anatomic pathology workflow. Another example could be establishing the relationship of the parent specimen (in this example tissue) to the specimen children output being created during the lab process, such as a block or a slide. The screenshot in FIG. 9 shows an example list of created System Object Maps. Some of these relationships may be pre-programmed by the company for basic anatomic pathology laboratory workflow. Other maps can be created by the user through the software application GUI to allow the user to create customized workflow operations dependent upon their lab workflow or setup. For an example, the user can create a custom workflow that sends a specific type of specimen after the microtome workstation to the de-cloaker workstation for processing IHC stains on tissue. These custom System Object Maps use N-number of input System Object ID's to create one output System Object ID that shows the next logical operation of moving the specimen to the next workflow or shows the next child succession of the System Object, such as specimen parent tissue to specimen child block. System Object Maps can be grouped into specific operations or functions.

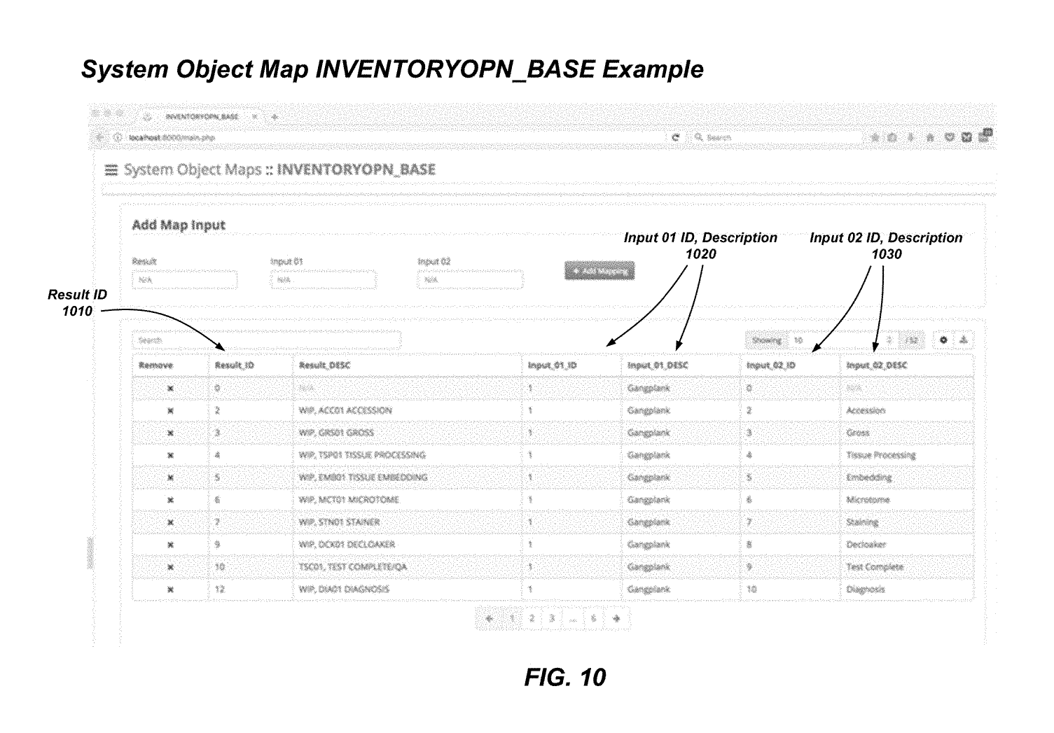

FIG. 10 shows a partial list of a System Object Map. In this example, the System Object Map is for an INVENTORYOPN_BASE, which is a fundamental operation being used to direct workflow of specimens in an anatomic pathology lab. The screenshot shows the first 10 of 52 map inputs being used. The software application shows columns representing a Remove operation (i.e., delete the particular System Object Map step), a Result_ID 1010, or output ID, of the System Object Map and the various input IDs with their associated description. Note that this System Object Map uses only 2 input IDs to process the workflow: Input_01_ID 1020 is set to the physical location (in this example the Gangplank Lab ENTITY location) and the next ID 1030, which is the processing step currently being used. This map is relating the ENTITY, or lab location, to the processing workstation steps in the lab.

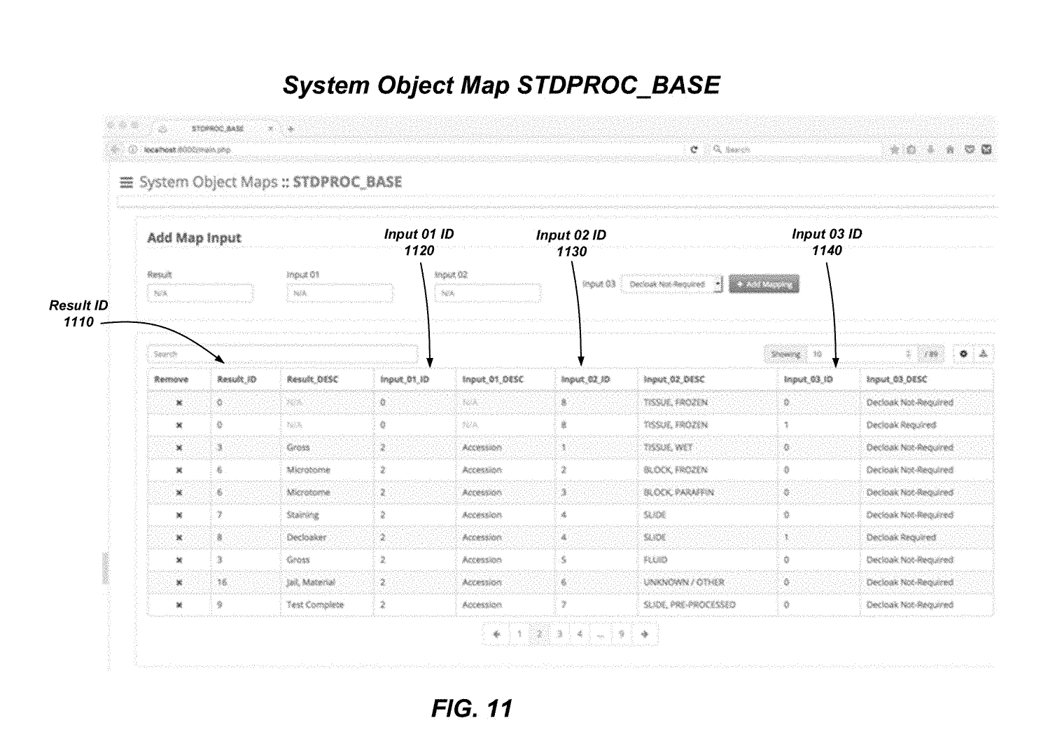

As another example, FIG. 11 shows a partial definition of a STDPROC_BASE System Object Map, which the software application uses to determine the next workstation step where the current Work-In-Process (WIP) material that is currently having work done at each workstation point in the lab needs to go. This map uses 3 ID inputs to direct the material from one workstation to another workstation in the lab. Input ID1 1120 shows the current workstation that is being referenced, Input ID2 1130 shows the material type and Input ID3 1140 shows if a de-cloak operation is required or not. The Result ID 1110 output callback value identifies the next workstation operation ID that should be referenced. The map engine logic (described above with reference to FIG. 2) of the software application uses each of these row inputs to do a comparison match to the current IDs being used at the current workstation step to determine the next workstation step.

In summary, embodiments of the present disclosure comprise a computing system for managing pathology lab workflow, which includes memory and one or more processors operably coupled to the memory. The memory stores a plurality of system objects, each system object representing an item to be tracked in the pathology lab workflow, the plurality of system objects including objects selected from the group consisting of accession, patient, and tissue samples. The memory also stores the computing instructions and a plurality of system object maps, each system object map designating transitions between operations being tracked within the pathology lab workflow. The one or more processors are configured for executing the computing instructions to perform a multi-relational analysis of two or more system objects of the plurality of system objects applied to one or more system object maps of the plurality of system object maps to identify a next state in the pathology lab for an item being tracked. The processors are also configured to output to a user the next state in the pathology lab for the item being tracked.

Embodiments of the present disclosure also include a computer-implemented method for managing pathology lab workflow. The method includes storing a plurality of system objects, each system object representing an item to be tracked in the pathology lab workflow, the plurality of system objects including objects selected from the group consisting of accession, patient, and tissue samples. The method also includes storing a plurality of system object maps, each system object map designating transitions between operations being tracked within the pathology lab workflow. The method also includes performing a multi-relational analysis of two or more system objects of the plurality of system objects applied to one or more system object maps of the plurality of system object maps to identify a next state in the pathology lab for an item being tracked and outputting to a user the next state in the pathology lab for the item being tracked.

Embodiments of the present disclosure further include a computer-implemented method for managing pathology lab workflow. The method includes configuring a relational database comprising a plurality of system objects maps, each system object map comprising two or more input identifiers and an output identifier. Each system object map is configured to define a relationship of the output identifier with the two or more input identifiers to designate at least one of a transition between operations being tracked within the pathology lab workflow and a relationship between a parent specimen and one or more children specimens. The method further includes performing a mapping process, which comprises receiving a plurality of input values representing an item to be tracked and searching the relational database. Searching the database identifies a specific system object map of the plurality wherein the plurality of input values corelate with the two or more input identifiers for the specific system object map and then either returns a map solution comprising the output identifier for the specific system object map or returns a non-solution value if searching the relational database did not identify any specific system object maps. Finally, the method includes outputting to a user a next state in the pathology lab for the item being tracked responsive to the map solution.

While the disclosure is susceptible to various modifications and implementation in alternative forms, specific embodiments have been shown by way of examples in the drawings and have been described in detail herein. It should be understood that the invention is not limited to the particular forms disclosed. Rather, the invention includes all modifications, equivalents, and alternatives falling within the scope of the following appended claims and their legal equivalents.

What is claimed is:

1. A computing system for managing pathology lab workflow, comprising:
memory configured to:
store a plurality of system objects, each system object representing an item to be tracked in the pathology lab workflow, the plurality of system objects including objects selected from the group consisting of accession, patient, and tissue samples;
store a plurality of system object maps, each system object map designating transitions between operations being tracked within the pathology lab workflow; and
store computing instructions; and
one or more processors operably coupled to the memory and configured for executing the computing instructions to:
perform a multi-relational analysis of two or more system objects of the plurality of system objects applied to one or more system object maps of the plurality of system object maps to identify a next state in the pathology lab for an item being tracked; and
output to a user the next state in the pathology lab for the item being tracked.

2. The computing system of claim 1, wherein the processing circuitry is further configured for executing the computing instructions to perform the multi-relational analysis using one or more-single layer map structures.

3. The computing system of claim 2, wherein the one or more single-layer map structures include a relationship selected from the group consisting of a one-to-one relational map, a conditional map, a list generation map, and an equivalency map.

4. The computing system of claim 1, wherein the processing circuitry is further configured for executing the computing instructions to perform the multi-relational analysis using one or more multi-layer map structures.

5. The computing system of claim 4, wherein the processing circuitry is further configured for executing the computing instructions to perform the multi-relational analysis using complex maps based on multiple input combinations of varying scope wherein the analysis comprises cascading through multiple layers of system object maps until a solution is found.

6. The computing system of claim 1, wherein the processing circuitry is further configured for executing the computing instructions to perform the multi-relational analysis using a combination of a single-layer map structure with an input condition of consistent scope and a multi-layer map structure with an input condition set with variations.

7. A computer-implemented method for managing pathology lab workflow, comprising:
storing a plurality of system objects, each system object representing an item to be tracked in the pathology lab workflow, the plurality of system objects including objects selected from the group consisting of accession, patient, and tissue samples;
storing a plurality of system object maps, each system object map designating transitions between operations being tracked within the pathology lab workflow; and
performing a multi-relational analysis of two or more system objects of the plurality of system objects applied to one or more system object maps of the plurality of system object maps to identify a next state in the pathology lab for an item being tracked; and
outputting to a user the next state in the pathology lab for the item being tracked.

8. The computer-implemented method of claim 7, further comprising performing the multi-relational analysis using one or more-single layer map structures.

9. The computer-implemented method of claim 8, wherein using the one or more single-layer map structures includes using a map structure with a relationship selected from the group consisting of a one-to-one relational map, a conditional map, a list generation map, and an equivalency map.

10. The computer-implemented method of claim 7, further comprising performing the multi-relational analysis using one or more multi-layer map structures.

11. The computer-implemented method of claim 10, further comprising performing the multi-relational analysis using complex maps based on multiple input combinations of varying scope wherein the analysis comprises cascading through multiple layers of system object maps until a solution is found.

12. The computer-implemented method of claim 7, further comprising performing the multi-relational analysis using a combination of a single-layer map structure with an input condition of consistent scope and a multi-layer map structure with an input condition set with variations.

13. A computer-implemented method for managing pathology lab workflow, comprising:
configuring a relational database comprising a plurality of system objects maps, each system object map comprising:
two or more input identifiers; and
an output identifier;
wherein each system object map is configured to define a relationship of the output identifier with the two or more input identifiers to designate at least one of a transition between operations being tracked within the pathology lab workflow and a relationship between a parent specimen and one or more children specimens; and
performing a mapping process comprising:
receiving a plurality of input values representing an item to be tracked; and
searching the relational database to:
identify a specific system object map of the plurality wherein the plurality of input values corelate with the two or more input identifiers for the specific system object map; and
return a map solution comprising the output identifier for the specific system object map or return a non-solution value if searching the relational database did not identify any specific system object maps; and
outputting to a user a next state in the pathology lab for the item being tracked responsive to the map solution.

14. The computer-implemented method of claim 13, wherein:
configuring the relational database further comprises including a plurality of system objects, representing an item to be tracked in the pathology lab workflow, the plurality of system objects including objects selected from the group consisting of accession, patient, and tissue samples; and
performing the mapping process further comprises deriving the plurality of input values from at least one of the plurality of system objects.

15. The computer-implemented method of claim 13, wherein performing the mapping process further comprises defining a process to include an enable list type allowing the mapping process to return multiple map solutions as a list.

16. The computer-implemented method of claim 13, wherein performing the mapping process further comprises defining a process to enable layer cascade continuation allowing the mapping process to return a map solution from a last solution found rather than the map solution from the first solution found.

17. The computer-implemented method of claim 13, wherein performing the mapping process further comprises defining a process to enable layer cascade continuation allowing the mapping process to return a map solution from a last solution found rather than the map solution from the first solution found.

18. The computer-implemented method of claim 13, wherein configuring the relational database further includes configuring one or more system object maps with a relationship selected from the group consisting of a one-to-one relational map, a conditional map, a list generation map, and an equivalency map.

19. The computer-implemented method of claim 13, wherein identifying a specific system object map comprises using a combination of a single-layer map structure with an input condition of consistent scope and a multi-layer map structure with an input condition set with variations.

20. The computer-implemented method of claim 13, wherein identifying a specific system object map comprises using complex maps based on multiple input combinations of varying scope and the identifying comprises cascading through multiple layers of system object maps until a solution is found.
